(19) United States
(12) Patent Application Publication
(10) Pub. No.: US 2026/0248970 A1
(43) Pub. Date: Aug. 27, 2026

(54) RADIOPAQUE AMINO-FUNCTIONAL POLY(AMINO ACID) COMPOUNDS FOR MEDICAL APPLICATIONS

(71) Applicant: Boston Scientific Scimed, Inc., Maple Grove, MN (US)

(72) Inventors: Philip David Dorgan, Cork (IE); Eddy Mateo Flores, Cork (IE); Cliodhna Wallace, Cork (IE); Rick William Dorn, Plymouth, MN (US); Joseph Thomas Delaney, JR., Minneapolis, MN (US); Cristian Parisi, Amherst, MA (US); Yen-Hao Hsu, Shrewsbury, MA (US); Lauren Sfakis Lydecker, Upton, MA (US)

(73) Assignee: Boston Scientific Scimed, Inc., Maple Grove, MN (US)

(21) Appl. No.: 19/548,211

(22) Filed: Feb. 24, 2026

Related U.S. Application Data
(60) Provisional application No. 63/762,728, filed on Feb. 25, 2025.

(51) Int. Cl. A61K 49/04; A61K 9/06; C08G 69/48

(52) U.S. Cl. CPC A61K 49/0442; A61K 9/06; C08G 69/48

(57) ABSTRACT

The present disclosure pertains to iodinated poly (amino acid) compounds that comprise residues of alpha amino acids that comprise an alpha carboxyl group, an alpha amino group, and a primary-amine-containing side chain comprising an amino group. The poly(amino acid) compounds comprise amide linkages formed from the alpha carboxyl groups and the amino groups of the primary-amine-containing side chains of the alpha amino acids forming the iodinated poly(amino acid) compound, at least one of the residues of the alpha amino acids in the iodinated poly(amino acid) compound is covalently modified to include one or more iodine atoms, and at least two of the alpha amino acid residues in the iodinated poly(amino acid) compound are not covalently modified. The present disclosure also provides crosslinked radiopaque hydrogels formed from such iodinated poly(amino acid) compounds, methods of treatment using such iodinated poly(amino acid) compounds, and systems containing such iodinated poly(amino acid) compounds.

### CROSS-REFERENCE TO RELATED APPLICATIONS

This application claims the benefit of U.S. Provisional Patent Application Ser. No. 63/762,728 filed on Feb. 25, 2025, the disclosure of which is incorporated herein by reference.

### FIELD

The present disclosure relates to radiopaque amino-functional poly(amino acid) compounds, to methods of forming radiopaque amino-functional poly(amino acid) compounds, to crosslinking agents containing radiopaque amino-functional poly(amino acid) compounds, and to medical hydrogels formed from crosslinking agents containing radiopaque amino-functional poly(amino acid) compounds.

BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically used biomaterial in prostate cancer therapy. A further improvement based on this structure is that a portion of the succinimidyl glutarate end groups have been replaced with 2,3,5-triiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups of SpaceOAR® and SpaceOAR Vue® will rapidly react with the trilysine crosslinker in vivo to form a hydrogel. The hydrogel breaks down in-vivo over the course of about ca. 6-9 months. The breakdown occurs primarily through the hydrolysis of the ester linkages on the glutarate groups.

The present disclosure provides an alternative to replacing a portion of the succinimidyl glutarate end groups of the multi-arm PEG polymer of SpaceOAR Vue® with 2,3,5-triiodobenzamide groups and provides an alternative to existing radiopaque crosslinkers, when radiopacity is desired.

### SUMMARY

In various aspects, the present disclosure pertains to iodinated poly(amino acid) compounds that comprise residues of alpha amino acids that comprise an alpha carboxyl group, an alpha amino group, and a primary-amine-containing side chain comprising an amino group. The poly(amino acid) compounds comprise amide linkages formed from the alpha carboxyl groups and the amino groups of the primary-amine-containing side chains of the alpha amino acids forming the iodinated poly(amino acid) compound, at least one of the residues of the alpha amino acids in the iodinated poly(amino acid) compound is covalently modified to include one or more iodine atoms, and at least two of the alpha amino acid residues in the iodinated poly(amino acid) compound are not covalently modified.

In some embodiments, the primary-amine-containing side chains of the alpha amino acids comprise between 1 and 10 carbon atoms. In some of these embodiments, the primary-amine-containing side chains of the alpha amino acids are of the formula —(CH₂)ₓNH₂, where x is an integer ranging from 1 to 10.

In some embodiments, the iodinated poly(amino acid) compound is selected from (a) an iodinated gamma-poly(2,4-diaminobutanoic acid) compound comprising 2,4-diaminobutanoic acid residues, in which at least one of the 2,4-diaminobutanoic acid residues is covalently modified to include one or more iodine atoms and at least two of the 2,4-diaminobutanoic acid residues are not covalently modified, (b) an iodinated delta-polyornithine compound comprising ornithine residues, in which at least one of the ornithine residues is covalently modified to include one or more iodine atoms and at least two of the ornithine residues are not covalently modified, (c) an iodinated epsilon-polylysine compound comprising lysine residues, in which at least one of the lysine residues is covalently modified to include one or more iodine atoms and at least two of the lysine residues are not covalently modified, and (d) an iodinated zeta-poly(2,7-diaminoheptanoic acid) compound comprising 2,7-diaminoheptanoic acid residues, in which at least one of the 2,7-diaminoheptanoic acid residues is covalently modified to include one or more iodine atoms and at least two of the 2,7-diaminoheptanoic acid residues are not covalently modified.

In some embodiments, which can be used in conjunction with the above aspects and embodiments, the iodinated poly(amino acid) compound contains between 10 and 50 of the alpha amino acid residues.

In some embodiments, which can be used in conjunction with the above aspects and embodiments, (a) iodinated poly(amino acid) compound of any of claims 1-5, wherein from 30% to 60%, of the total number of alpha amino acid residues in the compound are covalently modified to include one or more iodine atoms.

In some embodiments, which can be used in conjunction with the above aspects and embodiments, at least three of the residues of the alpha amino acids in the iodinated poly(amino acid) compound are covalently modified and at least three of the alpha amino acid residues in the iodinated poly(amino acid) compound are not covalently modified.

In some embodiments, which can be used in conjunction with the above aspects and embodiments, the covalently modified residues of the alpha amino acids comprise a covalently attached iodine containing moiety comprising one or more covalently attached iodine atoms. In some of these embodiments, the covalently attached iodine containing moiety is attached to the amino acid residue through a residue of the alpha-amino group.

In some embodiments, the covalently attached iodine containing moiety are attached to the amino acid residue through a linkage that comprises an amide group.

In some embodiments, the covalently attached iodine containing moiety is attached to the amino acid residue through a linkage that comprises an ether group, a C1-C10 alkyl group, and an amide group.

In some embodiments, the covalently attached iodine containing moiety is attached to the amino acid residue through a linkage that comprises two amide groups and a C1-C10 alkyl group.

In some embodiments, the iodine containing moiety comprises an iodinated aromatic moiety that comprises at least one monocyclic or multicyclic aromatic structure that is substituted with one, two, three, four, five, six or more iodine atoms. In some of these embodiments, the at least one monocyclic or multicyclic aromatic structure is further substituted with one or more additional groups that comprise one or more of the following: hydroxyl groups, groups that comprise one or more C1-C6 hydroxyalkyl groups, carboxylic acid groups, groups that comprise one or more C2-C6 carboxyalkyl groups, acetamido groups, groups that comprise one or more acetamido groups, or groups that comprise one or more C1-C6 alkyl groups.

In some embodiments, which can be used in conjunction with the above aspects and embodiments, the covalently modified residues of the alpha amino acids comprise a covalently attached quaternary amine group having a negatively charged iodine atom as a counterion. In some of these embodiments, a nitrogen atom of the covalently attached quaternary amine group corresponds to a nitrogen atom of the alpha-amino group of the amino acid residue. In some of these embodiments, the covalently attached quaternary amine group is a group of the formula —NR₃⁺ group where R is a C1-C6 alkyl group.

In other aspects, the present disclosure pertains to systems for forming hydrogels that comprise (a) a first composition comprising an iodinated poly(amino acid) compound in accordance with any of the above aspects and embodiments, and (b) a second composition comprising a reactive polymer comprising a plurality of hydrophilic polymer segments and a plurality of reactive moieties, wherein the plurality of reactive moieties are reactive with amino groups of the iodinated poly(amino acid) compound to form covalent crosslinks.

In some embodiments, the reactive polymer is a multi-arm polymer that comprises three or more polymer arms linked to a core region, each arm comprising one of the hydrophilic polymer segments and one of the reactive moieties. In some of these embodiments, the core region comprises a polyol residue and/or the hydrophilic polymer segments are selected from polyalkylene oxide segments, polyester segments, polyoxazoline segments, polydioxanone segments, and polypeptide segments.

In some embodiments, which can be used in conjunction with the above aspects and embodiments, the systems comprise a first composition that comprises the iodinated poly(amino acid) compound in a first container and a second composition that comprises the reactive polymer in a second container, wherein the first container and the second container are independently selected from vials and syringe barrels. In some of these embodiments, the first container is a syringe barrel and the second container is a vial.

In other aspects, the present disclosure provides crosslinked radiopaque hydrogels produced by systems that comprise (a) an iodinated poly(amino acid) compound in accordance with any of the above aspects and embodiments and (b) a reactive polymer comprising a plurality of hydrophilic polymer segments and a plurality of reactive moieties, wherein the reactive moieties are reactive with amino groups of the iodinated poly(amino acid) compound to form covalent bonds.

In further aspects, the present disclosure provides methods of treatment comprising administering to a subject a mixture that comprises (a) an iodinated poly(amino acid) compound in accordance with any of the above aspects and embodiments and (b) a reactive polymer comprising a plurality of hydrophilic polymer segments and a plurality of reactive moieties, wherein the reactive moieties are reactive with amino groups of the iodinated poly(amino acid) compound to form covalent bonds, wherein the mixture is administered under conditions such that the amino groups of the iodinated poly(amino acid) compound and the reactive moieties of the reactive polymer form covalent crosslinks after administration.

In accordance various aspects and embodiments of the present disclosure, iodinated polyamino compounds are formed, which are radiopaque, are relatively easy to synthesize, and are expected to be biologically well-tolerated.

The above and other aspects, embodiments, features and benefits of the present disclosure will be readily apparent from the following detailed description.

### BRIEF DESCRIPTION OF THE DRAWINGS

FIG. 1 schematically illustrates the formation of an iodinated poly(amino acid) compound having covalently attached iodine-containing moieties, in accordance with an embodiment of the present disclosure.

FIG. 2 schematically illustrates the formation of another iodinated poly(amino acid) compound having covalently attached iodine-containing moieties, in accordance with an embodiment of the present disclosure.

FIG. 3 schematically illustrates the formation of a further iodinated poly(amino acid) compound having covalently attached iodine-containing moieties, in accordance with an embodiment of the present disclosure.

FIG. 4 schematically illustrates the formation of yet another an iodinated poly(amino acid) compound having covalently attached iodine-containing moieties, in accordance with an embodiment of the present disclosure.

FIG. 5 schematically illustrates the formation of an iodinated poly(amino acid) compound having ionically bound iodide ions, in accordance with yet another embodiment of the present disclosure.

FIG. 6 schematically illustrates a delivery device, in accordance with an embodiment of the present disclosure.

FIG. 7 schematically illustrates a delivery device, in accordance with another embodiment of the present disclosure.

### DETAILED DESCRIPTION

In various aspects, the present disclosure provides iodinated poly(amino acid) compounds, which can be used as crosslinking agents for the formation of medical hydrogels, among other uses.

In some embodiments, the iodinated poly(amino acid) compounds comprise residues of alpha amino acids that comprise an alpha carboxyl group (which is covalently bound to the alpha carbon of the alpha amino acid), an alpha amino group (which is covalently bound to the alpha carbon of the alpha amino acid), and a primary-amine-containing side chain that comprises an amino group (which primary-amine-containing side chain is also covalently bound to the alpha carbon of the alpha amino acid). The poly(amino acid) compounds comprise amide linkages formed from the alpha carboxyl groups and the amino groups of the primary-amine-containing side chains of the alpha amino acids molecules forming the poly(amino acid) compounds, wherein at least one of the alpha amino acid residues in the iodinated poly(amino acid) compounds is covalently modified to comprise one or more iodine atoms and wherein at least two of the alpha amino acid residues in the iodinated poly(amino acid) compounds are not covalently modified.

In some embodiments, the primary-amine-containing side chains of the alpha amino acids comprise between 1 and 20 or more carbon atoms, for example, comprising anywhere 1 to 2 to 3 to 4 to 5 to 6 to 8 to 10 to 12 to 14 to 16 to 18 to 20 carbon atoms (in other words, the number carbon atoms in the primary-amine-containing side chains may range between any two of the preceding numerical values). In certain beneficial embodiments, the primary-amine-containing side chains of the alpha amino acids comprise between 1 and 10 carbon atoms, more typically between 2 and 8 carbon atoms.

In some embodiments, the primary-amine-containing side chains of the alpha amino acids are of the formula —(CH₂)ₓNH₂ where x is an integer ranging from 1 to 20 or more, for example, comprising anywhere 1 to 2 to 3 to 4 to 5 to 6 to 8 to 10 to 12 to 14 to 16 to 18 to 20 carbon atoms (in other words, integer x may range between any two of the preceding numerical values). In certain beneficial embodiments, integer x ranges between 1 and 10 carbon atoms, more typically between 2 and 8 carbon atoms.

In some embodiments, the iodinated poly(amino acid) compounds are selected from iodinated gamma-poly(2,4-diaminobutanoic acid) compounds, iodinated delta-polyornithine compounds, iodinated epsilon-polylysine compounds, and zeta-poly(2,7-diaminoheptanoic acid) compounds.

In this regard, the iodinated poly(amino acid) compounds may be selected from iodinated gamma-poly(2,4-diaminobutanoic acid) compounds (in which amide linkages are formed from the alpha-carbon carboxyl groups and the gamma-carbon amino groups of the 2,4-diaminobutanoic acid molecules forming the compounds), iodinated delta-polyornithine compounds (also referred to as iodinated delta-poly(2,5-diaminopentanoic acid) compounds) (in which amide linkages are formed from the alpha-carbon carboxyl groups and the delta-carbon amino groups of the ornithine molecules forming the compounds), iodinated epsilon-polylysine compounds (also referred to as iodinated epsilon-poly(2,6-diaminohexanoic acid) compounds) (in which amide linkages are formed from the alpha-carbon carboxyl groups and the epsilon-carbon amino groups of the lysine molecules forming the compounds), and iodinated zeta-poly(2,7-diaminoheptanoic acid) compounds (in which amide linkages are formed from the alpha-carbon carboxyl groups and the gamma-carbon amino groups of the 2,7-diaminoheptanoic acid molecules forming the compounds). At least one of the amino acid residues in the iodinated poly(amino acid) compounds (i.e., at least one of the 2,4-diaminobutanoic acid residues, at least one of the ornithine residues, at least one of the lysine residues, or at least one of the 2,7-diaminoheptanoic acid residues) is covalently modified to comprise one or more iodine atoms, and at least two of the amino acid residues in the iodinated poly(amino acid) compounds (i.e., at least two of the 2,4-diaminobutanoic acid residues, at least two of the ornithine residues, at least two of the lysine residues, or at least two of the 2,7-diaminoheptanoic acid residues) are not covalently modified.

In various embodiments, the above-described iodinated poly(amino acid) compounds contain a total number of amino acid residues (which includes both covalently unmodified amino acid residues and covalently modified amino acid residues) ranging anywhere from 3 to 100 or more amino acid residues. For example, such iodinated poly(amino acid) compounds may contain a total number of amino acid residues ranging anywhere from 3 to 4 to 5 to 6 to 7 to 8 to 9 to 10 to 12 to 15 to 20 to 25 to 30 to 35 to 40 to 45 to 50 to 60 to 70 to 80 to 90 to 100 amino acid residues (in other words, the total number of amino acid residues in the iodinated poly(amino acid) compounds may range between any two of the preceding numerical values). In some exemplary embodiments, the present disclosure provides iodinated poly(amino acid) compounds that have a total number of amino acid residues ranging anywhere from 15 to 50 amino acid residues. In certain exemplary embodiments, the present disclosure provides iodinated poly(amino acid) compounds that have a total number of amino acid residues ranging anywhere from 20 to 40 amino acid residues. In certain exemplary embodiments, the present disclosure provides iodinated poly(amino acid) compounds that have a total number of amino acid residues ranging anywhere from 25 to 35 amino acid residues.

In some embodiments, the number of modified amino acid residues in such compounds may range anywhere from 1 to 98 modified amino acid residues, for example, ranging anywhere from 1 to 2 to 3 to 4 to 5 to 6 to 7 to 8 to 9 to 10 to 12 to 15 to 20 to 25 to 30 to 40 to 50 to 60 to 70 to 75 to 80 to 85 to 88 to 90 to 91 to 92 to 93 to 94 to 95 to 96 to 97 to 98 modified amino acid residues, with the number of unmodified amino acid residues in such compounds ranging anywhere from 99 to 98 to 97 to 96 to 95 to 94 to 93 to 92 to 91 to 90 to 88 to 85 to 80 to 75 to 70 to 60 to 50 to 40 to 30 to 25 to 20 to 15 to 12 to 10 to 9 to 8 to 7 to 6 to 5 to 4 to 3 to 2 unmodified modified amino acid residues.

As previously noted, in certain beneficial embodiments, the iodinated poly(amino acid) compound may be iodinated epsilon-polylysine, which can be formed from unmodified epsilon-polylysine, shown here in uncharged form,

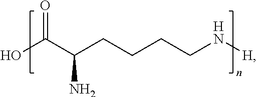

wherein n is an integer corresponding to the number of lysine residues and may range, for example, from 3 to 100 as indicated above. Epsilon-polylysine is a naturally occurring biopolymer that is readily soluble in water and is insoluble in non-polar organic solvents. Epsilon-polylysine and its derivatives have good heat stability and can undergo autoclave conditions without degradation, which can be beneficial in the context of medical applications such as medical hydrogels. Epsilon-polylysine may be susceptible to enzyme degradation, for instance, from Streptomyces peptidase.

The alpha amino (—NH₂) side groups of epsilon-polylysine provide reactive sites for covalent modification as described below. The same is true for the other poly(amino acid) compounds described herein, which comprise residues of alpha amino acids that comprise an alpha carboxyl group, an alpha amino group, and a primary-amine-containing side chain that comprises an amino group (which, along with the with alpha carboxyl groups, form amide linkages), including gamma-poly(2,4-diaminobutanoic acid), delta-polyornithine, and zeta-poly(2,7-diaminoheptanoic acid).

Epsilon-polylysine is also known to have antimicrobial properties. Therefore, iodinated epsilon-polylysine may exhibit antimicrobial properties as well. Where the iodinated epsilon-polylysine is used for form hydrogel compositions as described in more detail below, the epsilon-polylysine may add an inherent antimicrobial effect and achieve localized antimicrobial effect through surface-based interactions with microorganisms. In general, antimicrobial agents exhibit an ability to kill or inhibit the growth of microorganisms such as bacteria, viruses, fungi, and protozoa. Materials or compounds with antimicrobial properties can either destroy these microorganisms (bactericidal, viricidal, fungicidal) or suppress their ability to grow and reproduce (bacteriostatic, fungistatic). For instance, the antimicrobial properties of epsilon-polylysine (and potentially iodinated forms thereof) are manifested by altering the integrity and permeability of a microorganism's cell membrane through electrostatic interactions between the positively charged side chains of epsilon-polylysine and the negatively charged cell membrane. Following this carpet-like mechanism, it has been proposed that the lipopolysaccharide layer is removed by the epsilon-polylysine, which induces negative membrane curvature and leads to the formation of micelles and pores in the bacterial membrane, with the resulting permeability of the outer membrane leading to cell lysis. See, e.g., M. Hyldgaard et al. “The antimicrobial mechanism of action of epsilon-poly-l-lysine.” Appl Environ Microbiol. 2014 December; 80(24):7758-70.

As previously indicated, the iodinated poly(amino acid) compounds described herein (e.g., iodinated gamma-poly(2,4-diaminobutanoic acid) compounds, iodinated delta-polyornithine, iodinated epsilon-polylysine, and iodinated zeta-poly(2,7-diaminoheptanoic acid, among others) comprise two or more unmodified amino acid residues. Each of the unmodified amino acid residues provides an amino group along the polymer chain. The amino groups can either be in uncharged form (—NH₂) or in charged form (—NH₃⁺), depending, for example, on the pH. In instances where the amino groups are changed, various salt forms are possible including chloride, bromide, iodide, acetate, mesylate, maleate, formate, fumarate, tartrate, tosylate, phosphate and sulfate salts, among others.

The present disclosure also provides medical compositions that contain the above-described iodinated poly(amino acid) compounds. As detailed below, such compositions can be used in a system for forming a hydrogel that comprises (a) a first composition comprising an iodinated poly(amino acid) compound and (b) a second composition comprising a reactive polymer comprising a plurality of hydrophilic polymer segments and a plurality of reactive moieties, wherein the plurality of reactive moieties can react with unmodified amino groups of the iodinated poly(amino acid) compound to form covalent crosslinks.

In some embodiments, at least 75 wt %, at least 80 wt %, at least 85 wt %, at least 90 wt %, at least 95 wt %, at least 99 wt %, or 100% of the total amount of the iodinated poly(amino acid) compounds in the compositions may contain between 3and 100 amino acid residues. For example, at least 90 wt %, at least 95 wt %, at least 99 wt %, or 100% of the total weight of the iodinated poly(amino acid) compounds in the compositions may contain from 3 to 4 to 5 to 6 to 8 to 10 to 15 to 20 to 25 to 30 to 35 to 40 to 45 to 50 to 60 to 70 to 80 to 90 to 100 amino acid residues (i.e., ranging between any two of the preceding values).

In certain exemplary embodiments, the present disclosure provides compositions in which at least 90 wt %, at least 95 wt %, at least 99 wt %, or 100% of the total amount of the iodinated poly(amino acid) compounds in the compositions have a total number of amino acid residues ranging from 15 to 50 amino acid residues. In certain exemplary embodiments, the present disclosure provides compositions in which at least 90 wt %, at least 95 wt %, at least 99 wt %, or 100% of the total weight of the iodinated poly(amino acid) compounds in the compositions have a total number of amino acid residues ranging from 20 to 40 amino acid residues. In certain exemplary embodiments, the present disclosure provides compositions in which at least 90 wt %, at least 95 wt %, at least 99 wt %, or 100% of the total weight of the iodinated poly(amino acid) compounds in the compositions have a total number of amino acid residues ranging from 25 to 35 amino acid residues.

In some embodiments, anywhere from 1% to 99% of the total number of amino acid residues in the composition (i.e., modified and unmodified amino acid residues) are covalently modified and a remainder of the total number of amino acid residues in the composition are unmodified. For example, anywhere from 1% to 2% to 5% to 10% to 20% to 30% to 40% to 50% to 60% to 70% to 80% to 90% to 95% to 98% to 99% (i.e., ranging between any two of the preceding percentages) of the total number of amino acid residues in the composition may be covalently modified and a remainder of the total amino acid residues in the composition may be unmodified. In certain beneficial embodiments, from 20% to 70%, from 30% to 60%, or from 40% to 50% of the total number of amino acid residues in the composition may be covalently modified and a remainder of the total amino acid residues in the composition are unmodified.

In some embodiments, the compositions comprise a combination of iodinated poly(amino acid) compounds described herein with a non-iodinated polyamine compound. Where such a non-iodinated polyamine compound is provided, the weight ratio of iodinated poly(amino acid) compound to non-iodinated polyamine compound in the composition may range, for example, from 0.1:1 to 10:1, among other ratios.

Examples of non-iodinated polyamine compounds include poly(amino acid) compounds that comprise from 2 to 50 residues of basic alpha amino acids that comprise an alpha carboxyl group, an alpha amino group, and a primary-amine-containing side chain that comprises an amino group, such as 2,4-diaminobutanoic acid, ornithine, lysine, and 2,7-diaminoheptanoic acid. Such non-iodinated poly(amino acid) compounds may comprise amide linkages formed from the alpha carboxyl groups and the amino groups of the primary-amine-containing side chains of the basic alpha amino acids molecules forming the poly(amino acid) compounds, including as those described above, such as gamma-poly(2,4-diaminobutanoic acid), delta-polyornithine, epsilon-polylysine and zeta-poly(2,7-diaminoheptanoic acid), among others. Such non-iodinated poly(amino acid) compounds may comprise amide linkages formed from the alpha carboxyl groups and the alpha amino groups of the basic alpha amino acids molecules forming the poly(amino acid) compounds (as is the case with conventional poly(amino acid) compounds), such as alpha-poly(2,4-diaminobutanoic acid), alpha-polyornithine, alpha-polylysine and alpha-poly(2,7-diaminoheptanoic acid), generally simply known as poly(2,4-diaminobutanoic acid), polyornithine, polylysine and poly(2,7-diaminoheptanoic acid). A few particular examples of non-iodinated poly(amino acid) compounds include poly(amino acid) compounds that comprise from 2 to 10 lysine and/or ornithine amino-acid residues (e.g., dilysine, trilysine, tetralysine, pentalysine, diornithine, triornithine, tetraornithine, pentaornithine, etc.).

Non-iodinated polyamine compounds suitable for use in the present disclosure include those that comprise from 2 to 50 —(CH₂)ₓ—NH₂ groups where x is 0, 1, 2, 3, 4, 5 or 6.

Examples of non-iodinated polyamine compounds further include ethylenetriamine, diethylene triamine, hexamethylenetriamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, poly(vinyl amine), and poly(allyl amine), among others.

As previously noted, the present disclosure provides iodinated poly(amino acid) compounds wherein at least one of the alpha amino acid residues in the iodinated poly(amino acid) compounds is covalently modified to comprise one or more iodine atoms and wherein at least two of the alpha amino acid residues in the iodinated poly(amino acid) compounds are not covalently modified.

In various embodiments, the iodinated poly(amino acid) compounds of the present disclosure comprise at least one covalently modified amino acid residue that comprises at least one covalently attached iodine containing moiety.

In some embodiments, the covalently attached iodine containing moiety is attached to the amino acid residue through the alpha-amino group of the amino acid residue. In some embodiments, the covalently attached iodine containing moiety is attached to the amino acid residue through a linkage that comprises an amide group. In some embodiments, the covalently attached iodine containing moiety is attached to the amino acid residue through a linkage that comprises an ether group, a C1-C10 alkyl group, and an amide group. In some embodiments, the covalently attached iodine containing moiety is attached to the amino acid residue through a linkage that comprises two amide groups and a C1-C10 alkyl group.

In some embodiments, the iodine containing moiety is an iodinated aromatic moiety that comprises at least one monocyclic or multicyclic aromatic structure that is substituted with one, two, three, four, five, six or more iodine atoms. The at least one monocyclic or multicyclic aromatic structure can optionally be further substituted with one or more additional groups. Examples of additional groups include groups that comprise one or more hydroxyl groups including hydroxyl groups per se and groups that comprise one or more C1-C6 hydroxyalkyl groups, groups that comprise one or more carboxylic acid groups including carboxylic acid groups per se and groups that comprise one or more C2-C6 carboxyalkyl groups, include groups that comprise one or more acetamido groups including acetamido groups per se and groups that comprise one or more C1-C6 acetamido groups, and/or groups that comprise one or more C1-C6 alkyl groups, among others. Additional groups may be directly linked to the monocyclic or multicyclic aromatic structure or may be indirectly linked to the monocyclic or multicyclic aromatic structure through a suitable linkage, for example, a linkage that comprises one or more of the following: a C1-C6 alkyl group, an amide group, an ether group, a carbonate group, an ester group, a urea group, or a combination thereof.

In various embodiments, the iodinated poly(amino acid) compounds of the present disclosure comprise at least one covalently modified amino acid residue that comprises a covalently attached quaternary amine group, which has a negatively charged iodine atom as a counterion (also referred to herein as an iodine atom counterion).

In some embodiments, the covalently attached quaternary amine group corresponds to a modified alpha-amino group of the amino acid residue.

In some embodiments, the covalently attached quaternary amine group is an —NR₃⁺ group where R is a C1-C6 alkyl group (e.g., a methyl group, an ethyl group, an n-propyl group, an isopropyl group, an n-butyl group, an isobutyl group, a sec-butyl group, a tert-butyl group, an n-pentane group, another pentane isomer group, an n-hexane group, or another hexane isomer group.

Several methods for forming iodinated poly(amino acid) compounds that comprise at least one covalently modified amino acid residue that comprises a covalently attached iodine containing moiety comprising one or more covalently attached iodine atoms will now be described.

One way to form such iodinated poly(amino acid) compounds is through reaction of at least one unmodified alpha amino group of a poly(amino acid) compound as described herein. As indicated above such a poly(amino acid) compound comprises residues of alpha amino acids that comprise an alpha carboxyl group, an alpha amino group, and a primary-amine-containing side chain that comprises an amino group. Such a poly(amino acid) compound also comprises amide linkages formed from the alpha carboxyl groups and the amino groups of the primary-amine-containing side chains of the alpha amino acids molecules forming the poly(amino acid) compounds. Examples of such poly(amino acid) compounds include gamma-poly(2,4-diaminobutanoic acid), delta-polyornithine, epsilon-polylysine and zeta-poly(2,7-diaminoheptanoic acid) as described above.

In some embodiments, a carboxylic acid group of a carboxylic-acid-containing iodinated molecule is reacted in an amide coupling step with one or more amino groups of an unmodified poly(amino acid) compound (which may have a protected C-terminus carboxyl group as described below). Amino groups of the unmodified poly(amino acid) compound include the alpha amino groups that are present along the polymer backbone of the unmodified poly(amino acid) compound, as well as the amino group at the N-terminus of the poly(amino acid) compound, if unmodified.

The amide coupling step is typically performed in the presence of a suitable amide coupling agent. Examples of suitable amide coupling agents include carbodiimide coupling agents, such as N,N′-dicyclohexylcarbodiimide (DCC), 1-ethyl-3-(3-dimethyl′propyl) carbodiimide (EDC), 1,3-diisopropylcarbodiimide (DIC), N-hydroxybenzotriazole (HOBt), (benzotriazol-1-yloxy)tris(dimethylamino)phosphonium hexafluorophosphate (BOP reagent), and 2-(1H-Benzotriazole-1-yl)-1,1,3,3-tetramethylaminium tetrafluoroborate (TBTU), among others.

Protective groups may be provided as needed for any functional groups in the unmodified poly(amino acid compound) or the carboxylic-acid-containing iodinated molecule that can interfere with the amide coupling reaction between the carboxylic acid group of the carboxylic-acid-containing iodinated molecule and the amino groups of the iodinated poly(amino acid) compound. For example, in some embodiments, the carboxylic acid group at the C-terminus of the unmodified poly(amino acid compound), where unmodified, can be protected with a tert-butyl (tBu) ester group, which protects the carboxylic acid group, is acid labile, and can be removed, for example, with trifluoroacetic acid (TFA).

Examples of carboxylic-acid-containing iodinated compounds include those that comprise at least one iodinated aromatic group (e.g., a monocyclic or multicyclic aromatic structure that is substituted with one, two, three, four, five, six or more iodine atoms) and at least one carboxylic-acid-containing group. The carboxylic-acid-containing group may comprises for example, a carboxylic acid group and/or a C2-C6 carboxyalkyl group, which may be directly linked to the monocyclic or multicyclic aromatic structure or may be indirectly linked to the monocyclic or multicyclic aromatic structure through a linkage, for example, a linkage that comprises an amide group, an ether group, a carbonate group, an ester group, a urea group, or a combination thereof.

Specific examples of carboxylic-acid-containing iodinated molecule include the following, among many others: hydroxymethyl iodobenzoic acid,

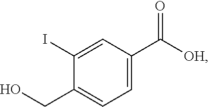 [SMILES: O=C(O)c1ccc(CO)c(I)c1]

hydroxymethyl diiodobenzoic acid,

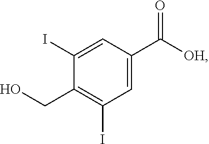 [SMILES: O=C(O)c1cc(I)c(CO)c(I)c1]

4,4-bis(hydroxy-3,5-diiodophenyl) pentanoic acid (IBHP),

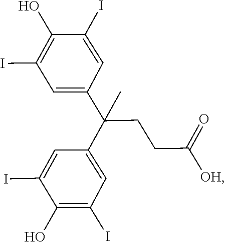 [SMILES: CC(CCC(=O)O)(c1cc(I)c(O)c(I)c1)c1cc(I)c(O)c(I)c1]

triiodobenzoic acid

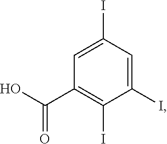 [SMILES: O=C(O)c1cc(I)cc(I)c1I]

diatrizoic acid,

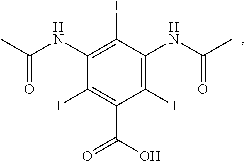 [SMILES: CC(=O)Nc1c(I)c(NC(C)=O)c(I)c(C(=O)O)c1I]

N-acetyl-3,5-diiodo-L-tyrosine,

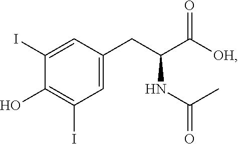 [SMILES: CC(=O)N[C@@H](Cc1cc(I)c(O)c(I)c1)C(=O)O]

N-acetyl-3-diiodo-L-tyrosine,

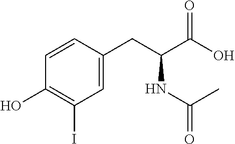 [SMILES: CC(=O)N[C@@H](Cc1ccc(O)c(I)c1)C(=O)O]

and N-acetyl-thyroxine,

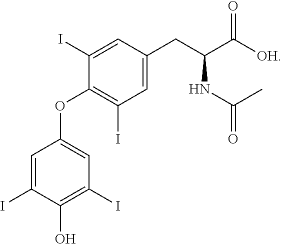 [SMILES: CC(=O)N[C@@H](Cc1cc(I)c(Oc2cc(I)c(O)c(I)c2)c(I)c1)C(=O)O]

Various carboxylic-acid-containing iodinated molecules, along with their CAS numbers, are listed in the following table:

| | | # I |
|---|---|---|
| Name | CAS | atoms |
| 2-Iodobenzoic acid | 88-67-5 | 1 |
| 4-Iodobenzoic acid | 619-58-9 | 1 |
| 3-Iodobenzoic acid | 618-51-9 | 1 |
| 4-Iodopicolinic acid | 405939-79-9 | 1 |
| 3,5-Diiodobenzoic acid | 19094-48-5 | 2 |
| 3,4-Diiodobenzoic acid | 35674-20-5 | 2 |
| 3,5-Diiodo-4-(4-methoxyphenoxy)benzoic acid | 34043-77-1 | 2 |
| Diatrizoate | 117-96-4 | 3 |
| 2,3,5-Triiodobenzoic acid | 88-82-4 | 3 |
| Acetrizoic acid | 85-36-9 | 3 |
| Metrizoate | 1949-45-7 | 3 |
| 3,4,5-Triiodobenzoic acid | 2338-20-7 | 3 |
| Ioxitalamic acid | 28179-44-4 | 3 |
| Iothalamic acid | 2276-90-6 | 3 |
| 2,4,6-Triiodo-1,3,5-benzenetricarboxylic acid | 79211-41-9 | 3 |
| 2,4,6-Triiodobenzoic acid | 2012-31-9 | 3 |
| Benzoic acid, 3-[[[2-Hydroxy-1- | 87932-11-4 | 3 |
| (hydroxymethyl)ethyl]amino]carbonyl]-5-[(2- | | |
| hydroxy-1-oxopropyl)amino]-2,4,6-triiodo-, | | |
| (S)- (9CI) | | |
| Ioseric acid | 51876-99-4 | 3 |
| 4-[4-(Acetyloxy)-3-iodophenoxy]-3,5- | 2260-084 | 3 |
| diiodobenzoic acid | | |
| Ioglicic acid | 49755-67-1 | 3 |
| 2,4,6-Triiodo-3-[(1-oxo-3,6,9,12,15- | 16024-67-2 | 3 |
| pentaoxahexadec-1-yl)amino]benzoic acid | | |
| 3-[[(1,1-Dimethylethoxy)carbonyl]amino]-2,4,6- | 2358047-48-8 | 3 |
| triiodobenzoic acid | | |
| 2,3,4,6-Tetraiodobenzoic acid | 71463-71-3 | 4 |
| Tetraform | 2055-97-2 | 4 |
| 2,3,5,6-Tetraiodo-1,4-benzenedicarboxylic acid | 7606-84-0 | 4 |
| N-Acetyl-O-(4-hydroxy-3,5-diiodophenyl)-3,5- | 26041-51-0 | 4 |
| diiodo-L-tyrosine | | |
| D-Tyrosine, N-[(1,1-dimethylethoxy)carbonyl]- | 89624-64-6 | 4 |
| O-(4-hydroxy-3,5-diiodophenyl)-3,5-diiodo- | | |
| (9CI, ACI) | | |
| 2,3,4,5,6-Pentaiodobenzoic acid | 64385-02-0 | 5 |
| Ioxaglic acid | 59017-64-0 | 6 |
| Ioglycamic acid | 2618-25-9 | 6 |
| Iocarmic acid | 10397-75-8 | 6 |

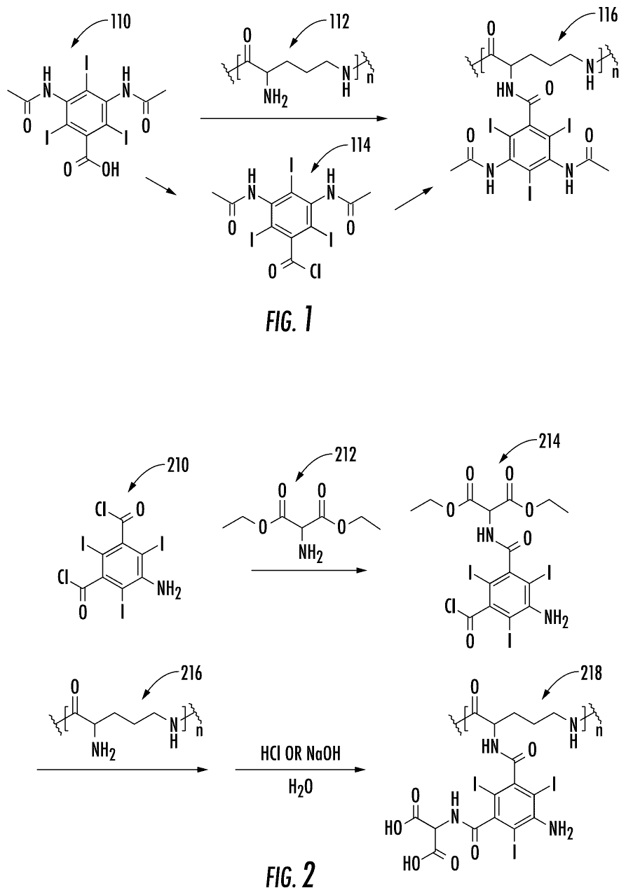

In a particular embodiment shown in FIG. 1, diatrizoate 110 is coupled with epsilon-polylysine 112 through two different reaction pathways.

In a first reaction pathway, the carboxylic acid group of diatrizoate 110 is directly coupled with an alpha amino group of the epsilon-polylysine 112 in an amide coupling reaction to form an iodinated epsilon-polylysine 116 in which one or more of the lysine residues are linked to diatrizoate residues through an amide group, and others of the lysine residues remain unmodified. Although not shown, at least two lysine residues in the iodinated epsilon-polylysine 116 remain unmodified. Such a coupling reaction is generally performed with a suitable amide coupling agent such as one of those described above. The ratio of modified lysine residues to unmodified residues in the resulting iodinated epsilon-polylysine compound 116 can be controlled by varying the ratio of the diatrizoate 110 to the epsilon-polylysine 112 in the reaction mixture.

In a second reaction pathway, the carboxylic acid group of diatrizoate 110 first converted to a more reactive acid chloride group. For example, the diatrizoate 110 may be reacted with thionyl chloride (SOCl₂) to form diatrizoic acid chloride 114. The diatrizoic acid chloride 114 is then reacted with epsilon-polylysine 112 in an amide coupling reaction to form an iodinated epsilon-polylysine 116 in which a portion of the lysine residues are linked to diatrizoate residues through an amide group. As noted above, although not shown, at least two lysine residues in the iodinated epsilon-polylysine 116 remain unmodified. The ratio of modified lysine residues to unmodified residues in the resulting iodinated epsilon-polylysine compound 116 can be controlled by varying the ratio of the diatrizoic acid chloride 114 to the epsilon-polylysine 112 in the reaction mixture. Because the acid chloride group of the diatrizoic acid chloride 114 is more reactive than the carboxylic acid group of the diatrizoate 110, the amide coupling reaction occurs spontaneously without the need for an amide coupling agent.

Examples of acid-chloride-containing iodinated compounds include those that comprise at least one iodinated aromatic group (e.g., a monocyclic or multicyclic aromatic structure that is substituted with one, two, three, four, five, six or more iodine atoms) and at least one acid-chloride-containing group. The acid-chloride-containing group may comprise for example, an acid chloride group and/or a C2-C6 alkyl acid chloride group, which may be directly linked to the monocyclic or multicyclic aromatic structure or may be indirectly linked to the monocyclic or multicyclic aromatic structure through a linkage, for example, a linkage that comprises an amide group, an ether group, a carbonate group, an ester group, a urea group, or a combination thereof.

Some specific examples of acid-chloride-containing iodinated compounds are found in the table below:

| | | # I |
|---|---|---|
| Name | CAS | atoms |
| 4-Iodobenzoyl chloride | 1711-02-0 | 1 |
| 2,3,5-Triiodobenzoyl chloride | 42860-33-3 | 3 |
| 5-Amino-2,4,6-triiodo-1,3-benzenedicarbonyl | 37441-29-5 | 3 |
| dichloride | | |
| 5-(Acetylmethylamino)-2,4,6-triiodo-1,3- | 79944-47-1 | 3 |
| benzenedicarbonyl dichloride | | |
| 5-(Acetylamino)-2,4,6-triiodo-1,3- | 31122-75-5 | 3 |
| benzenedicarbonyl dichloride | | |
| 5-[[2-(Acetyloxy)acetyl]amino]-2,4,6-triiodo-1,3- | 78314-12-2 | 3 |
| benzenedicarbonyl dichloride | | |
| 3,5-Bis(acetylamino)-2,4,6-triiodobenzoyl chloride | 103376-99-4 | 3 |
| 3-[[(2,3-Dihydroxypropyl)amino]carbonyl]-2,4,6- | 76350-04-4 | 3 |
| triiodo-5-[(2-methoxyacetyl)amino]benzoyl chloride | | |

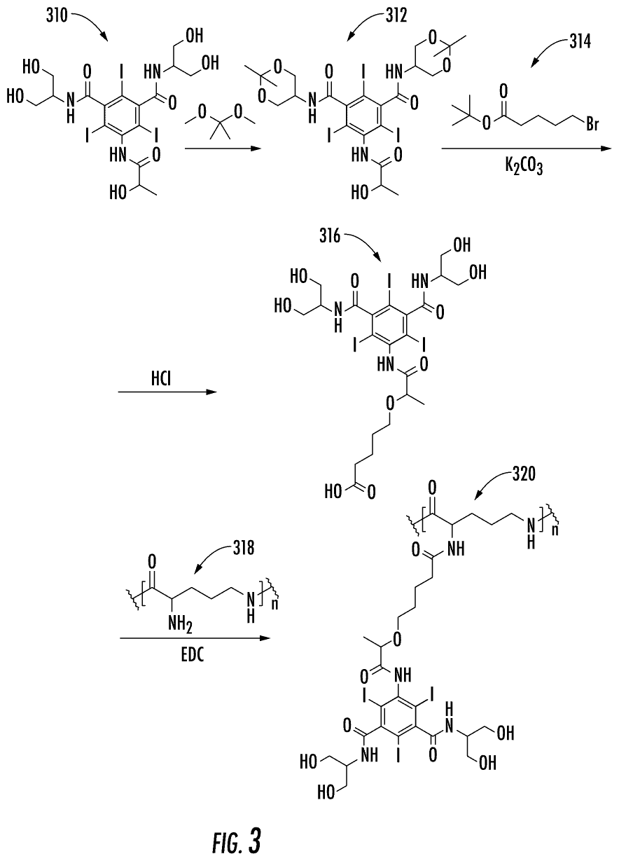

In embodiments where an iodinated compound having two or more acid chloride groups is employed in the formation of an iodinated poly(amino acid) compound, it may be desirable to reduce the number of acid chloride groups in the iodinated compound having two or more acid chloride groups to a single acid chloride group by reaction with an amine compound. With reference to FIG. 2, an stoichiometric excess of an iodinated compound having two acid chloride groups, specifically, 5-amino-2,4,6-triiodoisophthaloyl dichloride 210, is reacted with an amine compound, specifically, 3-amino-pentanedioic acid dimethyl ester 212 such that the amino group of the 3-amino-pentanedioic acid dimethyl ester 212 reacts in an amide coupling reaction with only one of the two acid chloride groups of the 5-amino-2,4,6-triiodoisophthaloyl dichloride 210. The resulting compound, diethyl 2-[(3-amino-5-chlorocarbonyl-2,4,6-triiodo-benzoyl)amino]propanedioate 214, is then reacted in an amide coupling reaction with epsilon-polylysine 216, followed by conversion of the methyl ester groups to carboxylic acid groups by exposure to acid or base (e.g., HCl or NaOH), to form an iodinated epsilon-polylysine 218 in which one or more of the lysine residues are linked to compound 214 residues through an amide group, and others of the lysine residues remain unmodified. Although not shown, at least two lysine residues in the iodinated epsilon-polylysine 218 remain unmodified. The ratio of modified lysine residues to unmodified residues in the resulting iodinated epsilon-polylysine 218 can be controlled by varying the ratio of the compound 214 to the epsilon-polylysine 218 in the reaction mixture.

In some embodiments, a carboxylic-acid-group-containing iodinated species may be formed from a hydroxyl-containing iodinated species by reacting a hydroxyl group of the hydroxyl-group-containing iodinated species with a halogen-containing carboxylic-acid compound in which the carboxylic acid group of the halogen-containing carboxylic-acid compound is protected. Such a halogen-containing carboxylic-acid compound may comprise, for example, a C1-C10 alkyl moiety (e.g., a methyl moiety, an ethyl moiety, an n-propyl moiety, an isopropyl moiety, an n-butyl moiety, an isobutyl moiety, a sec-butyl moiety, a tert-butyl moiety, an n-pentane moiety, other pentane isomer moieties, an n-hexane moiety, other hexane isomer moieties, an n-heptane moiety, other heptane isomer moieties, an n-octane moiety, other octane isomer moieties, etc.) substituted by a carboxylic-acid group and a halogen group (e.g., a bromine or iodine group). A few specific examples include haloacetic acid, 3-halopropanoic acid, 4-halobutanoic acid, 5-halopentanoic acid, 6-halohexanoic acid, 7-haloheptanoic acid, and 8-halooctanoic acid, among many others, where halo is bromo or iodo.

Hydroxyl-containing iodinated species include hydroxyalkyl-containing iodinated aromatic compounds. Examples of hydroxyalkyl-containing iodinated aromatic compounds include those that comprise one or more monocyclic or multicyclic aromatic groups, substituted with (a) one or more iodine groups (e.g., one two, three, four, five, six, seven, eight, nine, ten or more iodine atoms) and (b) one or more hydroxyl-containing groups independently selected from one or more hydroxyl groups and/or one or more C1-C4-hydroxyalkyl groups (e.g., C1-C4-monohydroxyalkyl groups, C1-C4-dihydroxyalkyl groups, C1-C4-trihydroxyalkyl groups, C1-C4-tetrahydroxyalkyl groups, etc.), among others, which C1-C4-hydroxyalkyl groups may be linked to the one or more monocyclic or multicyclic aromatic groups directly or through any suitable linking moiety, which may be selected, for example, from alkyl groups (e.g., alkyl groups containing one carbon, two carbons, three carbons, four carbons, etc.), amide groups, ether groups, carbonate groups, ester groups, urea groups, urethane groups, or combinations thereof, among others.

Particular examples of hydroxyl-containing iodinated species, several of which are commercially available, include the following, among others:

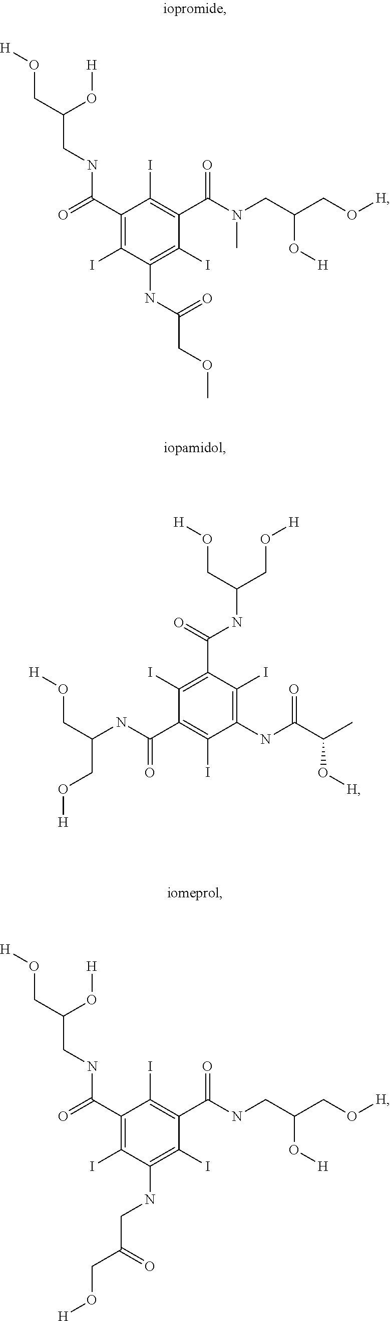

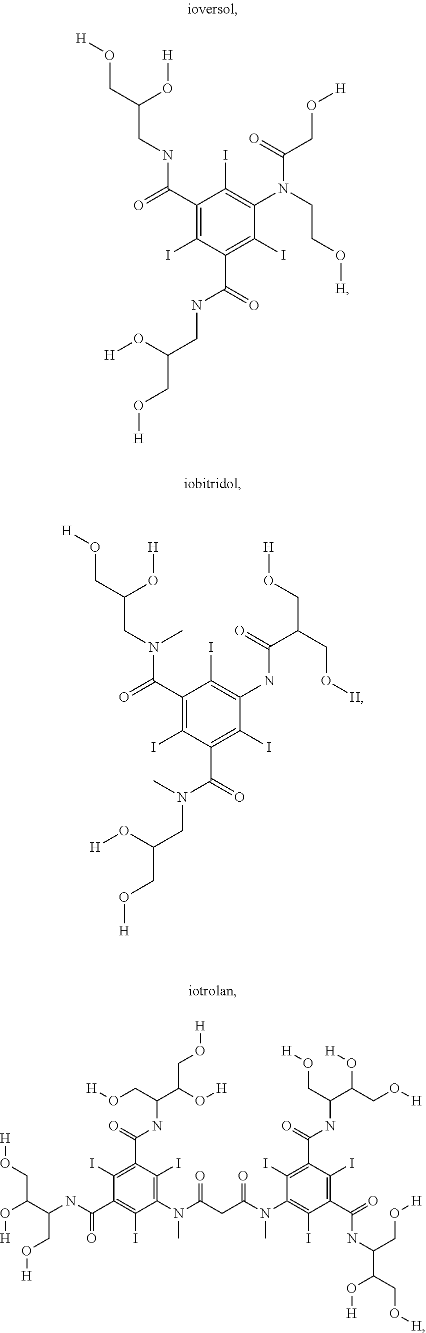

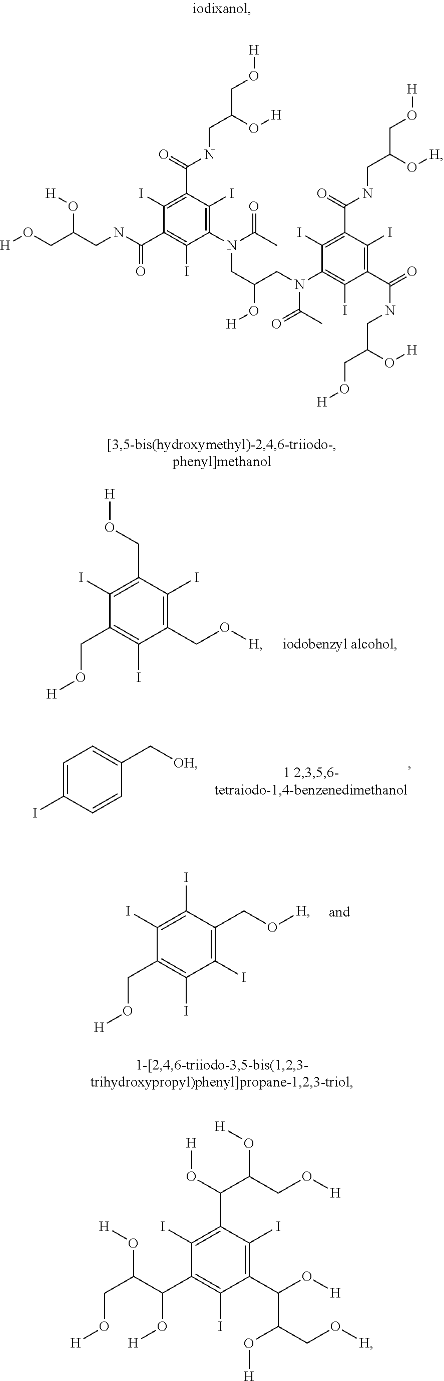

among others.

Further hydroxyalkyl-containing iodinated aromatic compounds are presented in the table below, along with their CAS numbers.

| | | # of OH |
|---|---|---|
| Compound | CAS# | Groups |
| Iopromide | 73334-07-3 | 4 |
| 5-Acetamido-N,N′-bis(2,3- | 31127-80-7 | 4 |
| dihydroxypropyl)-2,4,6-triiodoisophthalamide | | |
| Metrizamide | 31112-62-6 | 4 |
| 1,3-Benzenedicarboxamide,5-(acetylamino)- | 76350-28-2 | 4 |
| N1,N3-bis(2,3-dihydroxypropyl)-2,4,6-triiodo- | | |
| N1-methyl- (ACI) | | |
| Iopamidol | 60166-93-0 | 5 |
| Iomeprol | 78649-41-9 | 5 |
| Ioxaglate | 67992-58-9 | |
| 1,3-Benzenedicarboxamide,N1,N3-bis[2- | 60208-45-9 | 5 |
| hydroxy-1-(hydroxymethyl)ethyl]-5-[(2- | | |
| hydroxy-1-oxopropyl)amino]-2,4,6-triiodo- (ACI) | | |
| Ioxilan | 107793-72-6 | 5 |
| Iopentol | 89797-00-2 | 5 |
| Ioversol | 87771-40-2 | 6 |
| Iohexol | 66108-95-0 | 6 |
| Iobitridol | 136949-58-1 | 6 |
| Iodixanol | 92339-11-2 | 9 |
| 1,3-Benzenedicarboxamide, 5,5′-[(1,3-dioxo-1,3- | 79770-24-4 | 12 |
| propanediyl)bis(methylimino)]bis[N,N′-bis[2,3- | | |
| dihydroxy-1-(hydroxymethyl)propyl]-2,4,6- | | |
| triiodo- (9CI, ACI) | | |

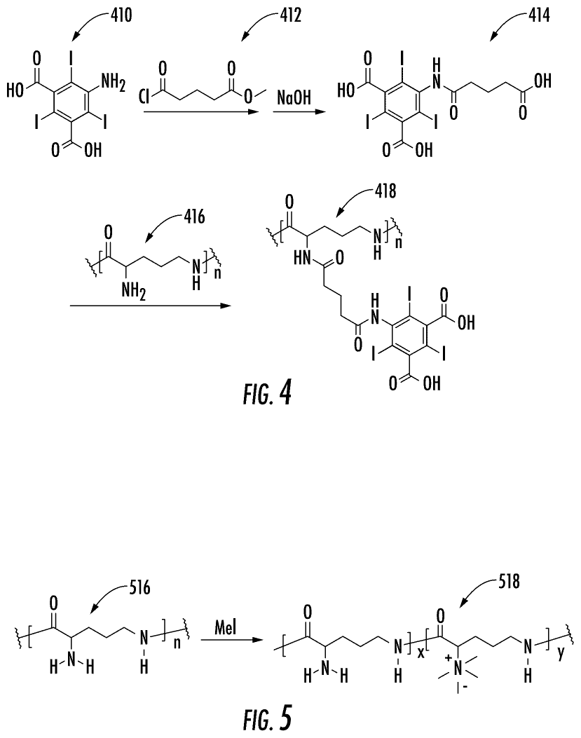

With reference now to FIG. 3, a hydroxyl-group-containing iodinated species, specifically, iopromide 310 is first reacted with 2,2-dimethoxypropane to form partially protected iopromide 312 in which the vicinal diol groups of the iopromide are protected by acetal groups and the remaining non-vicinal-diol hydroxyl group of the iopromide remains unprotected. The partially protected iopromide 312 is then reacted with a protected halogen-containing carboxylic-acid compound in which the carboxylic acid group has been protected, specifically, a t-butyl ester of 5-bromopentanoic acid 314, which is a halogen-containing carboxylic-acid compound that comprises an n-butyl alkyl moiety substituted with a protected carboxylic-acid group and a bromine group. The reaction is carried out in the presence of a strong base, such as potassium carbonate (K₂CO₃), followed by removal of the tert-butyl protection, for example, by exposure to an acid such as HCl or trifluoroacetic acid, to yield an iodinated carboxylic-acid-containing compound 316 in which a residue of the hydroxyalkyl-substituted iodinated aromatic compound, specifically a residue of iopromide, is linked to a C2-C11 carboxyalkyl group, specifically a carboxybutyl group, through an ether linkage.

The carboxylic acid group of the iodinated carboxylic-acid-containing compound 316 can then be used to couple the iodinated carboxylic-acid-containing compound 316 to alpha amino groups of a poly(amino acid) compound as described herein (e.g., epsilon-polylysine, gamma-poly(2,4-diaminobutanoic acid), delta-polyornithine, zeta-poly(2,7-diaminoheptanoic acid, etc.) via an amide coupling reaction as described above.

In the particular embodiment shown in FIG. 3, the carboxylic acid group of the iodinated carboxylic-acid-containing compound 316 is coupled with an alpha amino group of epsilon-polylysine 318 in an amide coupling reaction to form an iodinated epsilon-polylysine 320 in which one or more of the lysine residues are linked to iopromide residues through an ether group, a C1-C10 alkyl group (specifically a C4 alkyl group), and an amide group, and others of the lysine residues remain unmodified. The coupling reaction may be performed directly through the use of a suitable amide coupling agent such as one of those described above. The coupling reaction may also be performed by first converting the carboxylic acid group of the iodinated carboxylic-acid-containing compound 316 to a more reactive acid chloride group as described above (not shown), followed by reaction with epsilon-polylysine 318 in an amide coupling reaction to form an iodinated epsilon-polylysine 320 in which one or more of the lysine residues are linked to iopromide residues through an amide group, and others of the lysine residues remain unmodified. Although not illustrated, at least two lysine residues in the iodinated epsilon-polylysine 320 remain unmodified.

In some embodiments, a carboxylic-acid-group-containing iodinated species may be formed from an amino-containing iodinated species by reacting an amino group of the amino-group-containing iodinated species with a compound that comprise as acid chloride group and a protected carboxylic acid group, for example, a C1-C4-alkyl-ester-protected carboxylic acid group. Such a compound may comprise, for example, a C1-C10 alkyl moiety (e.g., a methyl moiety, an ethyl moiety, an n-propyl moiety, an isopropyl moiety, an n-butyl moiety, an isobutyl moiety, a sec-butyl moiety, a tert-butyl moiety, an n-pentane moiety, another pentane isomer moiety, an n-hexane moiety, another hexane isomer moiety, an n-heptane moiety, another heptane isomer moiety, an n-octane moiety, another octane isomer moiety, etc.) substituted by an acid chloride group and a protected carboxylic acid group, for example, a C1-C4-alkyl-ester-protected carboxylic acid group such as a methyl-ester-protected carboxylic acid group, an ethyl-ester-protected carboxylic acid group, an n-propyl-or isopropyl-ester-protected carboxylic acid group, an n-butyl, sec-butyl, isobutyl-or tertbutyl-ester-protected carboxylic acid group, and so forth. A few specific examples include 1,3-propanedioic acid monomethyl ester chloride (also known as malonic acid monomethyl ester chloride, in which a methyl moiety is substituted with an acid chloride group and a methyl-ester protected carboxyl group), 1,4-butanedioic acid monomethyl ester chloride (also known as succinic acid monomethyl ester chloride, in which an ethyl moiety is substituted at opposite ends with an acid chloride group and a methyl-ester protected carboxyl group), 1,5-pentanedioic acid monomethyl ester chloride (also known as glutaric acid monomethyl ester chloride, in which an n-propyl moiety is substituted at opposite ends with an acid chloride group and a methyl-ester protected carboxyl group), 1,6-hexanedioic acid monomethyl ester chloride (also known as adipic acid monomethyl ester chloride, in which an n-butyl moiety is substituted at opposite ends with an acid chloride group and a methyl-ester protected carboxyl group), 1,7-heptanedioic acid monomethyl ester chloride (also known as pimelic acid monomethyl ester chloride, in which an n-pentyl moiety is substituted at opposite ends with an acid chloride group and a methyl-ester protected carboxyl group), 1,8-octanedioic acid monomethyl ester chloride (also known as suberic acid monomethyl ester chloride, in which an n-heptyl moiety is substituted at opposite ends with an acid chloride group and a methyl-ester protected carboxyl group), among many others.

Examples of amino-containing iodinated species include those that comprise at least one iodinated aromatic group (e.g., a monocyclic or multicyclic aromatic structure that is substituted with one, two, three, four, five, six or more iodine atoms) and at least one amino-containing group. The amino-containing group may comprise for example, an amino group and/or a C2-C6 alkylamino group, which may be directly linked to the monocyclic or multicyclic aromatic structure or may be indirectly linked to the monocyclic or multicyclic aromatic structure through a linkage, for example, a linkage that comprises an amide group, ether group, a carbonate group, an ester group, a urea group, or a combination thereof.

Particular examples of amino-containing iodinated species are presented in the table below, along with their CAS numbers.

| | | # I |
|---|---|---|
| Name | CAS# | atoms |
| 4-Iodobenzylamine | 39959-59-6 | 1 |
| 5-Amino-N,N′-bis(2,3- | 76801-93-9 | 3 |
| dihydroxypropyl)-2,4,6- | | |
| triiodoisophthalamide | | |
| 3,5-Diiodobenzenamine | 35122-96-4 | 2 |
| 2,4,6-Triiodobenzenamine | 24154-37-8 | 3 |
| 5-Amino-2,4,6- | 35453-19-1 | 3 |
| triiodoisophthalic acid | | |

With reference now to FIG. 4, an amino group of an amino-group-containing iodinated compound, specifically, an amino group of 5-amino-2,4,6-triiodoisophthalic acid 410, is reacted in an amide coupling step with an acid chloride group of a compound that comprises an acid chloride group and a protected carboxylic acid group, specifically, an acid chloride group of glutaric acid monomethyl ester chloride 412, which comprises an n-propyl moiety substituted with a methyl protected carboxylic-acid group and an acid chloride group. This coupling reaction is followed by removal of the methyl ester protection, for example, by exposure to an acid such as HCl or trifluoroacetic acid, to yield an iodinated carboxylic-acid-containing compound 414 in which a residue of the amino-group-containing iodinated compound, specifically a residue of 5-amino-2,4,6-triiodoisophthalic acid, is linked to a C2-C11 carboxyalkyl group, specifically a carboxypropyl group, through an amide linkage.

The carboxylic acid group of the iodinated carboxylic-acid-containing compound 414 can then be used to couple the iodinated carboxylic-acid-containing compound 414 to amino groups of a poly(amino acid) compound as described herein (e.g., epsilon-polylysine, gamma-poly(2,4-diaminobutanoic acid), delta-polyornithine, zeta-poly(2,7-diaminoheptanoic acid, etc.) via an amide coupling reaction as described above. In the particular embodiment shown in FIG. 4, the carboxylic acid group of the iodinated carboxylic-acid-containing compound 414 is coupled with an amino group of epsilon-polylysine 416 in an amide coupling reaction to form an iodinated epsilon-polylysine 418 in which a portion of the lysine residues are linked to residues of the 5-amino-2,4,6-triiodoisophthalic acid through two amide groups and a C1-C10 alkyl group (specifically C4 alkyl group). The coupling reaction may be performed directly through the use of a suitable amide coupling agent such as one of those described above. The coupling reaction may also be performed by first converting the carboxylic acid group of the iodinated carboxylic-acid-containing compound 414 to a more reactive acid chloride group as described above, followed by reaction with epsilon-polylysine 416 in an amide coupling reaction to form an iodinated epsilon-polylysine 418 in which one or more of the lysine residues are linked to 5-amino-2,4,6-triiodoisophthalic acid residues through two amide groups and a C1-C10 alkyl group, and others of the lysine residues remain unmodified. Although not illustrated, at least two lysine residues in the iodinated epsilon-polylysine 418 remain unmodified.

As previously indicated, in some aspects, the iodinated poly(amino acid) compounds of the present disclosure comprise at least one covalently modified amino acid residue that comprises a covalently attached quaternary amine group having an iodine atom counterion. In some embodiments, the covalently attached quaternary amine group corresponds to a modification of the alpha-amino group of the amino acid residue. In some embodiments, the covalently attached quaternary amine group is an —NH₂R group where R is a C1-C6 alkyl group.

Methods will now be described for forming iodinated poly(amino acid) compounds that comprise at least one covalently modified amino acid residue that comprises a covalently attached quaternary amine group having an iodine atom counterion. In these methods, one or more of the alpha amino side groups of a poly(amino acid) compound as described herein (e.g., epsilon-polylysine, gamma-poly(2,4-diaminobutanoic acid), delta-polyornithine, zeta-poly(2,7-diaminoheptanoic acid, etc.) are converted to quaternary amine groups. In one embodiment, the one or more of the alpha amino side groups are reacted with an iodo-alkane (also referred to herein as an alkyl iodide). When the primary amine reacts with the iodo-alkane, it undergoes a number of nucleophilic substitution reactions, adding alkyl groups to the nitrogen atom until a quaternary amine is formed.

Iodoalkanes for use in the present disclosure include C1-C6 iodoalkanes (also referred to as C1-C6 alkyl iodides) including iodomethane, iodoethane, iodopropanes (e.g., 1-iodopropane, 2-iodopropane), iodobutanes (e.g., 1-iodobutane, 2-iodobutane, etc.), iodopentanes (e.g., 1-iodopentane, 2-iodopentane, 3-iodopentane, etc.), and iodohexanes (e.g., 1-iodohexane, 2-iodohexane, 3-iodohexane, etc.). Such iodoalkanes produce products having radiopaque iodine atom counteranions for the quaternary cations.

In a particular example shown in FIG. 5, iodomethane (MeI) is reacted with epsilon-polylysine 516 to form an iodinated epsilon-polylysine 518 in which a first portion of the alpha amino side groups of the lysine residues of the epsilon-polylysine 516, designated by the integer x, are converted into trimethylamino quaternary amine side groups with iodide counterions, and in which a second portion of the alpha amino side groups of the epsilon-polylysine 516, designated by the integer y, remain unmodified by the iodomethane. This can be accomplished by ensuring that there is a molar excess of the iodomethane relative to the moles of amino groups in the epsilon-polylysine. In some cases, monomethylamino and/or dimethylamino groups may also be present.

In further aspects of the present disclosure, crosslinked radiopaque hydrogels are provided that comprise a crosslinked reaction product of (a) an iodinated poly(amino acid) compound in accordance with the present disclosure and (b) a reactive polymer comprising reactive moieties that are reactive with the amino groups of the iodinated poly(amino acid) compound.

Reactive polymers for use in the present disclosure include reactive multi-arm polymers that comprise a plurality of polymer arms linked to a core region, wherein the polymer arms comprise a hydrophilic polymer segment. In some embodiments, a first end of the hydrophilic polymer segment is covalently linked to the core region and a reactive moiety is covalently linked to a second end (opposite end) of the hydrophilic polymer segment.

In some embodiments, the reactive moiety is covalently linked to the second end of the hydrophilic polymer segment through a cyclic anhydride residue or a lactone residue. For example, the second end of the hydrophilic polymer segment may be covalently linked to a first end of the cyclic anhydride or lactone residue and the reactive moiety may be covalently linked to a second end of the cyclic anhydride or lactone residue.

Reactive polymers in accordance with the present disclosure include multi-arm polymers having from 3 to 100 arms, for example, ranging anywhere from 3 to 4 to 5 to 6 to 7 to 8 to 9 to 10 to 11 to 12 to 15 to 20 to 25 to 50 to 75 to 100 arms (in other words, ranging between any two of the preceding numerical values).

Reactive moieties include reactive moieties that comprise electrophilic groups and reactive moieties that comprise unsaturated groups.

Electrophilic groups may be selected, for example, from cyclic imide ester groups, such as succinimide ester groups,

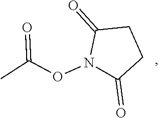

maleimide ester groups, glutarimide ester groups, diglycolimide ester groups, phthalimide ester groups, and bicyclo[2.2.1] hept-5-ene-2,3-dicarboxylic acid imide ester groups,

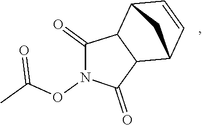 [SMILES: CC(=O)ON1C(=O)C2C(C1=O)[C@H]1C=C[C@@H]2C1]

imidazole ester groups, imidazole carboxylate groups and benzotriazole ester groups, among other possibilities.

Unsaturated groups may be selected, for example, from unsaturated end groups having double carbon-carbon bonds such as acrylate ester groups and unsaturated end groups having triple carbon-carbon bonds such as propiolate ester groups.

The electrophilic or unsaturated groups may be linked to the hydrophilic polymer segment through any suitable linking moiety, which may be selected, for example, from a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups, among others. In certain embodiments, the linking moiety comprises a hydrolysable ester group.

Hydrophilic polymer segments for the polymer arms can be selected from a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain between 5 and 1000 monomer units or more, for example, ranging anywhere from 5 to 10 to 20 to 50 to 100 to 200 to 500 to 1000 to 2000 monomer units.

In certain embodiments, the core region comprises a residue of a polyol comprising three or more hydroxyl groups, which is used to form the polymer arms. For example, the core region may comprise a residue of a polyol that contains from 3 to 100 hydroxyl groups, for example, ranging anywhere from 3 to 4 to 5 to 6 to 7 to 8 to 9 to 10 to 11 to 12 to 15 to 20 to 25 to 50 to 75 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, tetrapentaerythritol, adonitol, hexaglycerol. dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl)cresols, among others.

Illustrative polyols also include calixarenes such as, for example, calix[n]arenes where n is 4, 5, 6, 7, 8, 9,10, 11, 12, or more, among other possibilities.

Illustrative polyols further include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 4 to 5 to 6 to 8 to 10 to 25 to 50 to 100 monomer units in length.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

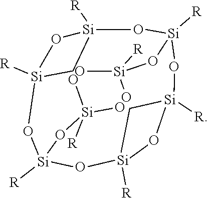

In the present disclosure, the R groups comprise the polymer arms described herein.

Reactive multi-arm polymers in accordance with the present disclosure can be formed from hydroxyl-terminated precursor multi-arm polymers having arms that comprise one or more hydroxyl end groups.

In some of these embodiments, the hydroxyl-terminated precursor multi-arm hydrophilic polymer may be reacted with a cyclic anhydride to form an acid-end-capped precursor polymer. For example, terminal hydroxyl groups of the hydrophilic segments may be reacted with a cyclic anhydride (e.g., a glutaric anhydride compound, a succinic anhydride compound, a malonic anhydride compound, an adipic anhydride compound, a diglycolic anhydride compound, etc.) to form an acid-end-capped segment such as a glutaric-acid-end-capped segment, a succinic-acid-end-capped segment, a malonic-acid-end-capped segment, an adipic-acid-end-capped segment, a diglycolic-acid-end-capped segment, and so forth.

The preceding cyclic anhydrides, among others, may be reacted with a hydroxyl-terminated precursor multi-arm hydrophilic polymer under basic conditions to form a carboxylic-acid-terminated precursor polymer comprising a carboxylic acid end group that is linked to a hydrophilic polymer segment through a hydrolysable ester group.

A reactive moiety may then be linked to the carboxylic-acid-terminated precursor polymer.

In some embodiments, an electrophilic moiety may be linked to the carboxylic-acid-terminated precursor polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl′propyl)carbodiimide (EDC), N-hydroxybenzotriazole (HOBt), BOP reagent, and/or another coupling agent) to form a reactive cyclic imide ester (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

Reactive multi-arm polymers having reactive moieties that comprise unsaturated groups can also be formed from hydroxy-terminated precursor multi-arm polymers having arms that comprise one or more hydroxyl end groups.

In some embodiments, an unsaturated moiety may be linked a hydroxyl-terminated precursor multi-arm polymer. In a particular example, a reactive multi-arm polymer may be formed by reacting acryloyl chloride with hydroxyl end groups of a hydroxyl-terminated precursor multi-arm polymer, thereby forming unsaturated acrylate ester groups at the sites previously occupied by the hydroxyl groups. In one embodiment, a reactive multi-arm polymer, which comprises a core region and a plurality of polyethylene oxide (PEO) arms having reactive acrylate end groups is formed by reacting acryloyl chloride with hydroxyl end groups of a hydroxyl-terminated multi-arm polymer having a core region that comprises a polyol residue and multiple hydroxyl-terminated polyethylene oxide arms. In a particular example, a commercially available hydroxyl-terminated 8-arm PEG having a tripentaerythritol polyol residue core is reacted with acryloyl chloride to create an acrylate-terminated 8-arm PEO. Acrylate-terminated 8-arm PEO (also referred to acrylate-terminated 8-arm PEG) having a tripentaerythritol residue core and acrylate-terminated 4-arm PEO (also referred to an acrylate-terminated 4-arm PEG) having a pentaerythritol residue core are also available from JenKem Technology USA (Plano, Texas, USA).

In some embodiments, it is desirable to include a hydrolysable ester group in order to enhance the biodegradability of the reactive multi-arm polymer via hydrolysis. For example, hydroxyl end groups of a hydroxyl-terminated precursor multi-arm polymer may be reacted in a ring-opening reaction with a cyclic-ester-containing compound, specific examples of which include lactone compounds such as β-propiolactone, γ-butyrolactone, δ-valerolactone, and ε-caprolactone, among other lactones, thereby forming a multi-arm polymer having hydroxyalkyl end groups, for example, C₁-C₆-hydroxyalkyl end groups, which are linked to the polymer arm through an ester group. In other words, a hydroxyl-terminated multi-arm polymer is formed in which polymer arms are end capped with a hydroxyalkyl ester group, such as a hydroxymethyl ester group, a hydroxyethyl ester group, a hydroxypropyl ester group, a hydroxybutyl ester group, etc. In a particular embodiment, terminal hydroxyl groups of a hydroxyl-terminated multi-arm polymer, for example, a hydroxyl-terminated 8-arm PEG) having a core region that comprises a polyol residue, for example, a tripentaerythritol residue, and multiple hydroxyl-terminated polyethylene oxide arms, for example, eight hydroxyl-terminated polyethylene oxide arms, are reacted with a cyclic-ester-containing compound, in particular, ε-caprolactone, in a ring-opening reaction forming a hydroxybutyl-ester-end-capped multi-arm PEG. This hydroxyl-terminated polymer may then be reacted with acryloyl chloride as described above, thereby forming unsaturated acrylate ester groups at the sites previously occupied by the hydroxyl groups.

As previously noted, in some aspects, the present disclosure provides crosslinked radiopaque hydrogels that comprise a crosslinked reaction product of (a) an iodinated poly(amino acid) compound in accordance with the present disclosure and (b) a reactive polymer comprising reactive moieties that are reactive with amino groups of the iodinated poly(amino acid) compound.

In some embodiments, reaction between reactive moieties of the reactive polymer that comprise electrophilic groups, specifically cyclic imide groups, and the amino groups of the iodinated poly(amino acid) compound is conducted at slightly basic pH (e.g., having a pH value ranging from 7.4 to 11) where the amino groups of the iodinated poly(amino acid) compound are deprotonated/neutrally charged and amide bond formation can occur spontaneously at room or body temperature between the cyclic imide groups and the amino groups.

In other embodiments, an iodinated poly(amino acid) compound as described herein is reacted with a reactive hydrophilic polymer having reactive moieties that comprise unsaturated groups, for example, an eight-arm PEG with reactive acrylate ester groups having a core region that comprises a tripentaerythritol residue like that described above. The reactive hydrophilic polymer and the iodinated poly(amino acid) compound may be combined under conditions such that the unsaturated groups of the reactive hydrophilic polymer react with the primary amine groups of iodinated poly(amino acid) compound via Michael addition to form amide bonds, thereby forming a crosslinked polymer network. In certain embodiments, reaction between the unsaturated groups and the amino groups is conducted at slightly basic pH (e.g., having a pH value ranging from 7.4 to 11) where the amino groups of the iodinated poly(amino acid) compound are deprotonated/neutrally charged and the Michael addition can occur spontaneously at room or body temperature.

It will be appreciated that the crosslinking density of the resulting crosslinked radiopaque hydrogels can be tuned, for example, (a) by varying the number of primary amine groups in the iodinated poly(amino acid) compound, (b) by varying the number of arms of the reactive multi-arm polymer, or (c) both.

Such crosslinked radiopaque hydrogels may be formed in vivo (e.g., using a delivery device like that described below), or such crosslinked radiopaque hydrogels may be formed ex vivo and subsequently administered to a subject. Such crosslinked radiopaque hydrogels can be used in a variety of biomedical applications, including implants, medical devices, and pharmaceutical compositions.

In some embodiments, the crosslinked radiopaque hydrogel is visible under various X-ray imaging techniques. The crosslinked radiopaque hydrogel may have a radiopacity that is greater than 100 Hounsfield units (HU), beneficially ranging anywhere from 100 HU to 250 HU to 500 HU to 750 HU to 1000 HU or more (in other words, ranging between any two of the preceding numerical values), for example, when measured on bench-top micro CT systems such as XtremeCT from Scanco Medical (Wangen-Brüttisellen, Switzerland) or similar.

In some aspects of the present disclosure, a system is provided that comprises (a) a first composition that comprises an iodinated poly(amino acid) compound as described herein and (b) a second composition that comprises a reactive polymer comprising reactive moieties as described herein, wherein the system is configured to deliver the reactive polymer and the iodinated poly(amino acid) compound under conditions such that covalent crosslinks are formed between the reactive polymer and the iodinated poly(amino acid) compound.

The first composition may be a first fluid composition comprising the iodinated poly(amino acid) compound or a first dry composition that comprises the iodinated poly(amino acid) compound, to which a suitable fluid such as water for injection, saline, etc. can be added to form a first fluid composition. In addition to the iodinated poly(amino acid) compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the iodinated poly(amino acid) compound with a second fluid comprising the reactive polymer. Upon mixing the first and second fluid compositions, the iodinated poly(amino acid) compound crosslinks with the reactive polymer, forming a crosslinked product. The first and second fluid compositions may be combined form crosslinked radiopaque hydrogels, either in vivo or ex vivo.

In some embodiments, the iodinated poly(amino acid) compound is initially combined with the reactive polymer under conditions where crosslinking between the reactive polymer and the iodinated poly(amino acid) compound is suppressed (e.g., an acidic pH). Then, when crosslinking is desired, the conditions are changed such that crosslinking is increased (e.g., a change from an acidic pH to a basic pH, in some embodiments), leading to crosslinking between the iodinated poly(amino acid) compound and the reactive polymer, thereby forming a crosslinked product. The first and second fluid compositions may be combined form crosslinked radiopaque hydrogels, either in vivo or ex vivo.

In some embodiments, the system comprises (a) a first composition that comprises an iodinated poly(amino acid) compound as described hereinabove, (b) a second composition that comprises a reactive polymer as described hereinabove, and (c) a third composition, specifically, an accelerant composition, that contains an accelerant that is configured to accelerate a crosslinking reaction between the iodinated poly(amino acid) compound and the reactive polymer.

The first composition may be a first fluid composition comprising the iodinated poly(amino acid) compound that is buffered to an acidic pH or a first dry composition that comprises the iodinated poly(amino acid) compound, to which a suitable fluid such as water for injection, saline, an acidic buffer solution, etc. can be added to form a first fluid composition comprising the iodinated poly(amino acid) compound that is buffered to an acidic pH. In some embodiments, for example, the acidic buffering composition may comprise monobasic sodium phosphate, among other possibilities. The first fluid composition comprising the iodinated poly(amino acid) compound may have a pH ranging, for example, from about 3 to about 5. In addition to the iodinated poly(amino acid) compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the iodinated poly(amino acid) compound that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the iodinated poly(amino acid) compound that is buffered to an acidic pH and the second composition comprises a dry composition that comprises the reactive polymer. The first composition may then be mixed with the second composition to provide a prepared fluid composition that is buffered to an acidic pH and comprises the iodinated poly(amino acid) compound and the reactive polymer. In a particular example, a syringe may be provided that contains the first fluid composition comprising the iodinated poly(amino acid) compound that is buffered to an acidic pH, and a vial may be provided that comprises the dry composition (e.g., a powder) that comprises the reactive polymer. The syringe may then be used to inject the first fluid composition into the vial containing the reactive polymer to form a prepared fluid composition that is buffered to an acidic pH and contains the iodinated poly(amino acid) compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 8 to 12. In addition to the above, the fluid accelerant composition may further comprise additional agents, including those described below.

A prepared fluid composition that is buffered to an acidic pH and comprises the iodinated poly(amino acid) compound and the reactive polymer as described above, and a fluid accelerant composition that is buffered to basic pH as described above, may be combined form crosslinked radiopaque hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents (in addition to the iodinated poly(amino acid) compound), for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises an iodinated poly(amino acid) compound as described herein and a second reservoir that contains a second fluid composition that comprises a reactive polymer as described herein, wherein the first and second fluid compositions form a crosslinked product upon mixing.

In some embodiments, the system may include a delivery device that comprises a first reservoir that contains a first fluid composition that comprises the iodinated poly(amino acid) compound and the reactive polymer and is buffered to an acidic pH, such as the prepared fluid composition previously described, and a second reservoir that contains a second fluid composition, such as the fluid accelerant composition previously described.

During operation, the first fluid composition and the second fluid composition are dispensed from the first and second reservoirs and combined, whereupon the iodinated poly(amino acid) compound and the reactive polymer and crosslink with one another to form a crosslinked radiopaque hydrogel.

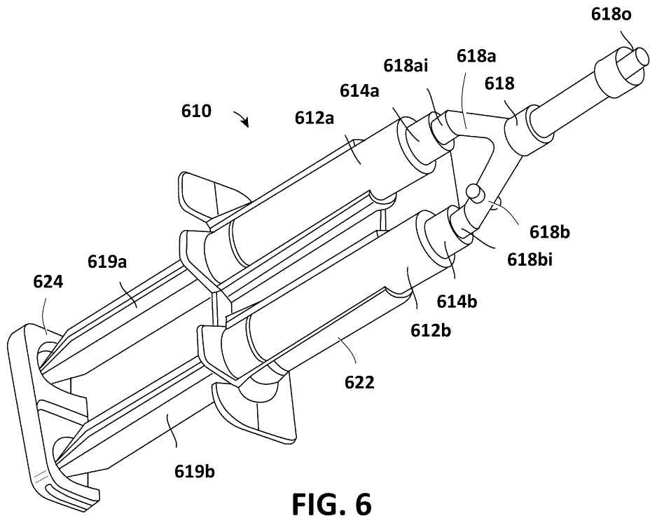

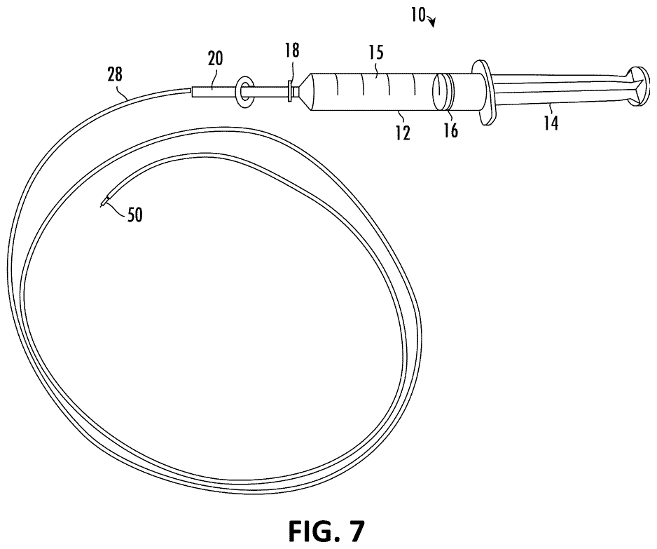

In particular embodiments, and with reference to FIG. 6, the system may include a delivery device 610 that comprises a double-barrel syringe, which includes a first barrel 612a having a first barrel outlet 614a, which first barrel contains a first fluid composition as described above, a first plunger 619a that is movable in the first barrel 612a, a second barrel 612b having a second barrel outlet 614b, which second barrel 612b contains a second fluid composition as described above, and a second plunger 619b that is movable in the second barrel 612b. In some embodiments, the device 610 may further comprise a mixing section 618 having a first mixing section inlet 618ai in fluid communication with the first barrel outlet 614a, a second mixing section inlet 618bi in fluid communication with the second barrel outlet 614b, and a mixing section outlet 618o. Also shown are a syringe holder 622 configured to hold the first and second syringe barrels 612a, 612b, in a fixed relationship and a plunger cap 624 configured to hold the first and second plungers 619a, 619b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked radiopaque hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked radiopaque hydrogel is ultimately formed at the administration location.

During and/or after administration, the compositions of the present disclosure can be imaged using a suitable imaging technique. Typically, the imaging technique is an x-ray-based imaging technique, such as computerized tomography or x-ray fluoroscopy, or a near near-IR fluorescence spectrometry-based technique.

The first and second fluid compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions or fluid admixtures of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked radiopaque hydrogels may be in any desired form, including a slab, a cylinder, a coating, or a particle. In some embodiments, the crosslinked radiopaque hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked radiopaque hydrogel particles formed using the above and other techniques may vary widely in size, for example, having an average size ranging from 50 to 950 microns.

In addition to a crosslinked radiopaque hydrogel as described above, ex vivo crosslinked radiopaque hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the ex vivo crosslinked radiopaque hydrogel composition to a subject. Such systems may include one or more of the following: a syringe barrel, which may or may not contain a crosslinked radiopaque hydrogel composition as described herein; a vial, which may or may not contain a crosslinked radiopaque hydrogel composition as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked radiopaque hydrogel composition may be provided in dry form (e.g., powder form) or in a form that is ready for injection, such as an injectable hydrogel form (e.g., a suspension of crosslinked radiopaque hydrogel particles).

FIG. 7 illustrates a syringe 10 providing a reservoir for a crosslinked radiopaque hydrogel composition as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked radiopaque hydrogel composition 15 for injection through the needle 50.

The ex vivo crosslinked radiopaque hydrogel compositions described herein can be used for a number of purposes. For example, crosslinked radiopaque hydrogel compositions can be injected to provide spacing between tissues, crosslinked radiopaque hydrogel compositions can be injected (e.g., in the form of blebs) to provide fiducial markers, crosslinked radiopaque hydrogel compositions can be injected for tissue augmentation or regeneration, crosslinked radiopaque hydrogel compositions can be injected as a filler or replacement for soft tissue, crosslinked radiopaque hydrogel compositions can be injected to provide mechanical support for compromised tissue, crosslinked radiopaque hydrogel compositions be injected as a scaffold, and/or crosslinked radiopaque hydrogel compositions can be injected as a carrier of therapeutic agents in the treatment of diseases and cancers and the repair and regeneration of tissue, among other uses.

Ex vivo crosslinked radiopaque hydrogel compositions of the present disclosure may be used in a variety of medical procedures, including the following, among others: a procedure to implant a fiducial marker comprising a crosslinked radiopaque hydrogel, a procedure to implant a tissue regeneration scaffold comprising a crosslinked radiopaque hydrogel, a procedure to implant a tissue support comprising a crosslinked radiopaque hydrogel, a procedure to implant a tissue bulking agent comprising a crosslinked radiopaque hydrogel, a procedure to implant a therapeutic-agent-containing depot comprising a crosslinked radiopaque hydrogel, a tissue augmentation procedure comprising implanting a crosslinked radiopaque hydrogel, a procedure to introduce a crosslinked radiopaque hydrogel between a first tissue and a second tissue to space the first tissue from the second tissue.

Ex vivo crosslinked radiopaque hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Ex vivo crosslinked radiopaque hydrogel compositions in accordance with the present disclosure include lubricious compositions for medical applications, compositions for therapeutic agent release (e.g., by including one or more therapeutic agents in a matrix of the crosslinked radiopaque hydrogel), and implants (which may be formed ex vivo or in vivo) (e.g., compositions for use as tissue markers, compositions that act as spacers to reduce side effects of off-target radiation therapy, cosmetic compositions, etc.).

During and/or after administration, the ex vivo crosslinked radiopaque hydrogel compositions of the present disclosure can be imaged using a suitable imaging technique. Typically, the imaging technique is an x-ray-based imaging technique, such as computerized tomography or x-ray fluoroscopy, or a near near-IR fluorescence spectrometry-based technique.

1. An iodinated poly(amino acid) compound comprising residues of alpha amino acids that comprise an alpha carboxyl group, an alpha amino group, and a primary-amine-containing side chain comprising an amino group, wherein the poly(amino acid) compound comprises amide linkages formed from the alpha carboxyl groups and the amino groups of the primary-amine-containing side chains of the alpha amino acids forming the iodinated poly(amino acid) compound, wherein at least one of the residues of the alpha amino acids in the iodinated poly(amino acid) compound is covalently modified to include one or more iodine atoms and wherein at least two of the alpha amino acid residues in the iodinated poly(amino acid) compound are not covalently modified.

2. The iodinated poly(amino acid) compound of claim 1, wherein the primary-amine-containing side chains of the alpha amino acids comprise between 1 and 10 carbon atoms.

3. The iodinated poly(amino acid) compound of claim 1, wherein the primary-amine-containing side chains of the alpha amino acids are of the formula —(CH₂)ₓNH₂, where x is an integer ranging from 1 to 10.

4. The iodinated poly(amino acid) compound of claim 1, wherein the iodinated poly(amino acid) compound is selected from (a) an iodinated gamma-poly(2,4-diaminobutanoic acid) compound comprising 2,4-diaminobutanoic acid residues, in which at least one of the 2,4-diaminobutanoic acid residues is covalently modified to include one or more iodine atoms and at least two of the 2,4-diaminobutanoic acid residues are not covalently modified, (b) an iodinated delta-polyornithine compound comprising ornithine residues, in which at least one of the ornithine residues is covalently modified to include one or more iodine atoms and at least two of the ornithine residues are not covalently modified, (c) an iodinated epsilon-polylysine compound comprising lysine residues, in which at least one of the lysine residues is covalently modified to include one or more iodine atoms and at least two of the lysine residues are not covalently modified, and (d) an iodinated zeta-poly(2,7-diaminoheptanoic acid) compound comprising 2,7-diaminoheptanoic acid residues, in which at least one of the 2,7-diaminoheptanoic acid residues is covalently modified to include one or more iodine atoms and at least two of the 2,7-diaminoheptanoic acid residues are not covalently modified.

5. The iodinated poly(amino acid) compound of claim 1, wherein the iodinated poly(amino acid) compound contains between 10 and 50 of the alpha amino acid residues.

6. The iodinated poly(amino acid) compound of claim 1, wherein from 30% to 60%, of the total number of alpha amino acid residues in the compound are covalently modified to include one or more iodine atoms.

7. The iodinated poly(amino acid) compound of claim 1, wherein at least three of the residues of the alpha amino acids in the iodinated poly(amino acid) compound are covalently modified and at least three of the alpha amino acid residues in the iodinated poly(amino acid) compound are not covalently modified.

8. The iodinated poly(amino acid) compound of claim 1, wherein the covalently modified residues of the alpha amino acids comprise a covalently attached iodine containing moiety comprising one or more covalently attached iodine atoms.

9. The iodinated poly(amino acid) compound of claim 8, wherein the covalently attached iodine containing moiety is attached to the amino acid residue through a residue of the alpha-amino group.

10. The iodinated poly(amino acid) compound of claim 8, wherein the covalently attached iodine containing moiety is attached to the amino acid residue through a linkage that comprises an amide group, wherein the covalently attached iodine containing moiety is attached to the amino acid residue through a linkage that comprises an ether group, a C1-C10 alkyl group, and an amide group, or wherein the covalently attached iodine containing moiety is attached to the amino acid residue through a linkage that comprises two amide groups and a C1-C10 alkyl group.

11. The iodinated poly(amino acid) compound of claim 8, wherein the iodine containing moiety comprises an iodinated aromatic moiety that comprises at least one monocyclic or multicyclic aromatic structure that is substituted with one, two, three, four, five, six or more iodine atoms.

12. The iodinated poly(amino acid) compound of claim 11, wherein the at least one monocyclic or multicyclic aromatic structure is further substituted with one or more additional groups that comprise one or more of the following: hydroxyl groups, groups that comprise one or more C1-C6 hydroxyalkyl groups, carboxylic acid groups, groups that comprise one or more C2-C6 carboxyalkyl groups, acetamido groups, groups that comprise one or more acetamido groups, or groups that comprise one or more C1-C6 alkyl groups.

13. The iodinated poly(amino acid) compound of claim 1, wherein the covalently modified residues of the alpha amino acids comprise a covalently attached quaternary amine group having a negatively charged iodine atom as a counterion.

14. The iodinated poly(amino acid) compound of claim 13, wherein a nitrogen atom of the covalently attached quaternary amine group corresponds to a nitrogen atom of the alpha-amino group of the amino acid residue.

15. The iodinated poly(amino acid) compound of claim 13, wherein the covalently attached quaternary amine group is a group of the formula —NR₃⁺ group where R is a C1-C6 alkyl group.

16. A system for forming a hydrogel that comprises (a) a first composition comprising an iodinated poly(amino acid) compound that comprises residues of alpha amino acids that comprise an alpha carboxyl group, an alpha amino group, and a primary-amine-containing side chain comprising an amino group, wherein the poly(amino acid) compound comprises amide linkages formed from the alpha carboxyl groups and the amino groups of the primary-amine-containing side chains of the alpha amino acids forming the iodinated poly(amino acid) compound, wherein at least one of the residues of the alpha amino acids in the iodinated poly(amino acid) compound is covalently modified to include one or more iodine atoms and wherein at least two of the alpha amino acid residues in the iodinated poly(amino acid) compound are not covalently modified and (b) a second composition comprising a reactive polymer comprising a plurality of hydrophilic polymer segments and a plurality of reactive moieties, wherein the plurality of reactive moieties are reactive with amino groups of the iodinated poly(amino acid) compound to form covalent crosslinks.

17. The system of claim 16, wherein the reactive polymer is a multi-arm polymer that comprises three or more polymer arms linked to a core region, each arm comprising one of the hydrophilic polymer segments and one of the reactive moieties.

18. The system of claim 17, wherein the core region comprises a polyol residue and/or wherein the hydrophilic polymer segments are selected from polyalkylene oxide segments, polyester segments, polyoxazoline segments, polydioxanone segments, and polypeptide segments.

19. The system of claim 16, comprising a first composition that comprises the iodinated poly(amino acid) compound in a first container and a second composition that comprises the reactive polymer in a second container, wherein the first container and the second container are independently selected from vials and syringe barrels.

20. A method of treatment comprising administering to a subject a mixture that comprises (a) an iodinated poly(amino acid) compound that comprises residues of alpha amino acids that comprise an alpha carboxyl group, an alpha amino group, and a primary-amine-containing side chain comprising an amino group, wherein the poly(amino acid) compound comprises amide linkages formed from the alpha carboxyl groups and the amino groups of the primary-amine-containing side chains of the alpha amino acids forming the iodinated poly(amino acid) compound, wherein at least one of the residues of the alpha amino acids in the iodinated poly(amino acid) compound is covalently modified to include one or more iodine atoms and wherein at least two of the alpha amino acid residues in the iodinated poly(amino acid) compound are not covalently modified and (b) a reactive polymer comprising a plurality of hydrophilic polymer segments and a plurality of reactive moieties, wherein the reactive moieties are reactive with amino groups of the iodinated poly(amino acid) compound to form covalent bonds, wherein the mixture is administered under conditions such that the amino groups of the iodinated poly(amino acid) compound and the reactive moieties of the reactive polymer form covalent crosslinks after administration.
